(19) United States
(12) Patent Application Publication
(10) Pub. No.: US 2010/0240990 A1
(43) Pub. Date: Sep. 23, 2010

(54) Diagnosis and treatment method of malignant tumours and marker compound

(76) Inventor: Besiki Surguladze, Thornhill (CA)

Correspondence Address: GEORGE A. ROLSTON, 45 SHEPPARD AVE EAST, SUITE 900, TORONTO, ON, M2N5W9, CA

(21) Appl. No.: 12/382,628

(22) Filed: Mar. 19, 2009

(51) Int. Cl. A61B 6/00; A61B 8/00

(52) U.S. Cl. 600/431; 600/437

(57) ABSTRACT

A method of diagnosis and treatment of malignant tumours by the steps locating an area containing of lymph systems including lymph nodes and lymph ducts draining the area around a tumour, injecting a magnetic particles containing marker compound in and around the tumour, the compound migrating into the lymph system and lymph nodes draining the area around said tumour; making images of such area, by methods such as X-ray, Ultrasound and the like and identifying locations of lymph nodes, conducting biopsies of lymph nodes responding to the marker compound to determine the tumour status of such lymph nodes, and operating to remove both the malignant tumour and tumour-invaded lymph nodes, and subsequently subjecting the area around said tumour and containing any remaining said lymph nodes and ducts to an hyperthermal treatment by an oscillating electromagnetic field thereby inducing heat in said magnetic particles containing marker compound, to break down any potentially remaining tumour cells in the said remained micro lymph nodes in that area. Also disclosed is a marker compound for the marking , detecting, and possible subsequent treatment of the lymph system draining the tumour area.

### FIELD OF THE INVENTION

The invention relates to the diagnosis and treatment of malignant tumors, by contrasting and performing X ray or ultrasound control trepan biopsy, or biopsy of colored sentinel nodes of the lymph systems around malignant tumors, prior to starting an operation to remove the tumour, performing lymphadenectomy of coloured lymphatic nodes, and subjecting the regional lymph system area around the tumour area, subsequent to the removal of the tumour to hyperthermal treatment.

### BACKGROUND OF THE INVENTION

Medical treatment of malignant tumours have been proposed by various methods, including surgical removal, chemotherapy, radiation therapy, hyperthermal treatment, cryogenic therapy, implanted active pellets, sometimes radioactive, and other methods. The success or otherwise of these methods varies widely. One of the problems often encountered in therapy for malignant tumors, is that the cells of the malignant tumour have migrated into the lymph system surrounding the malignant tumour, known as “metastases”.

Lymph nodes are aggregates of lymphoid tissue contained within an encapsulated structure located along lymphatic vessels.

Malignant tumour cells often spread from the primary tumor to the nearest lymph node or nodes. Metastasis to the regional nodes is the single most important prognostic factor in solid neoplasms. If the cancer has spread the lymph node first to receive the material from an area of the tumor is called the sentinel lymph node. It is important to understand that the sentinel node will probably be the first one to get cells of the malignant tumor in it if the cells have has spread to the lymph nodes at all. Prior to introduction of intraoperative lymphatic mapping and sentinel lymphadenectomy, the only way to identify nodal metastases was to remove all regional nodes. However, this can cause troublesome postoperative complications and is of no benefit in most patients with clinically normal lymph nodes. Intraoperative lymphatic mapping and sentinel lymphadenectomy represents a minimally invasive but highly effective alternative. This surgical sampling technique identifies and examines the first (“sentinel”) lymph node on the drainage pathway from a malignant tumor. They found that if this node is free of tumor cells, then the primary malignant tumor is very unlikely to have spread from its site to regional lymph nodes. Patients whose sentinel node contains no evidence of tumor do not need to undergo further lymph node surgery.

Removal of any lymph nodes invaded by malignant tumor cells is sometimes included as part of the surgical operation to remove a tumour. However, this involves making biopsies of lymph nodes, while the operation is in progress. Biopsy samples must then be checked in a laboratory to determine the status of the nodes. This means that the operation must be suspended part way through, with the patient still under sedation, while the lab tests the samples. When the test results returned to the surgeon contain information about the presence of malignant tumor cells, he can then remove all lymph nodes of the lymphatic basin drainage area of the malignant tumor, and finally complete the operation. This delay is undesirable. Sometime it in itself is a separate surgical operation, and consumes valuable time of staff and facilities. It places excess stress on the patient, by prolonging the operation.

Cabanas [1] introduced the concept of the “sentinel node” in 1977 when he used lymphangiograms performed via dorsal lymphatics of the penis to demonstrate the existence group of specific nodes associated with the superficial epigastric vein that predicted the nodal status of penile carcinoma. In 1992, Morton and colleagues [2] described lymphatic mapping utilizing an intradermal isosulfan blue dye injection technique for melanoma and were the first to employ this concept to localize sentinel nodes in patients with melanoma. The authors demonstrated a high success rate in identifying a sentinel node and in achieving low false-negative rate. In 1993, Alex and Krag [3] introduced the use of a radioactive tracer ^{99m}Technetium sulphur colloid, injected intradermally around a primary-melanoma site, followed by imaging and subsequent intraoperative use of a gamma probe to localize and extirpate the sentinel node. In recent years, lymphoscintigraphy and blue dye have been used to trace the regional sentinel node in many tumours.

Sentinel lymph node biopsy is currently used in lieu of complete node dissection in malignant tumors. The technique used to detect sentinel nodes has focused on using a vital blue dye, a radioactive tracer, or a combination of both.

Isosulfan blue (trade mark) is the currently the dye of choice in North America; it is injected around the tumor minutes before node biopsy [4]. Massaging the injection site and exploring the nodal basin for visual identification of blue lymphatic channels leading to the blue lymph node follow the injection. Vital dyes are poorly retained in the node, and the color fades after twenty to thirty minutes, decreasing the detection rate [5].

The radiotracer technique uses a radioactive compound, usually ^{99m}Technetium, attached to a carrier such as sulfur colloid, albumin, or antimony [6]. This tracer is most frequently injected around the tumor 1 to 24 hours before surgery. In the operating room, a gamma probe detector, which emits a high-pitched sound when it detects radioactivity, is used to locate the sentinel lymph node. It is usually done with the support of an experienced nuclear medicine department.

Identification of the sentinel node is crucial for the success of the procedure. Although there is debate about the optimal method for sentinel node detection, many surgeons favor combining the techniques to improve the detection rate and facilitate the procedure [7, 11].

The combination of techniques may be associated with some disadvantages. The radiotracer is usually injected an hour before surgery and very frequently the night before surgery, creating a logistical inconvenience to patients and surgeons, particularly in busy centers [12, 13]. Although some have reported 90% of sentinel nodes being blue and radioactive with the combination technique [14], others have shown a concordance rate of blue and radioactive nodes in only 30% of the nodes [15].

Various problems have been reported with these techniques:

The Shine Through phenomenon. As reported by Albertini et al. this occurs when the original activity site or the tumor is too close to the axilla thus interfering with the axillary reading of the radiolabeled tracer.

Inner Quadrant Tumors. Inner quadrant tumors are not amenable to sentinel node lymphadenectomy for obvious location reasons. The lymphatic drainage of these tumors is usually via the internal mammary nodes.

Lymphatic Node Skipping Tumor Progression: There have been numerous concerns about skipped metastasis. Some authors have claimed it occurs in 15% of the cases. Albertini et al. in their study shows this does not affect the sentinel node mapping.

It is also the practice in malignant tumor therapy to administer further therapy after completion of the operation to remove the tumour, This post operative therapy has involved various modalities, such as radiation, chemo therapy, hyperthermal therapy, to name a few. However once the operation is completed it may be difficult to map out areas which may be targets for metastases, and to treat only those areas. As a result such therapies may possibly be administered with little or no accurate direction as to which are areas to target for possible metastases.

Clearly it is desirable to overcome both the problems of pre operative “mapping” of possible lymph node invasion, to enable removal of as much as possible of such organs, and also post operative isolation of invaded lymph systems which it was not possible to remove during the operation, for specific follow up therapy.

### BRIEF SUMMARY OF THE INVENTION

With a view to providing a solution to both of these problems the invention provides a method of diagnosis and treatment of malignant tumor and comprising the steps of, first locating an area containing of lymph systems including lymph nodes and lymph ducts, around a malignant tumor, injecting a magnetic particles containing marker compound in or around said tumour, said compound migrating into said lymph nodes around said tumour, making X-ray images or make images using other technology such as ultrasound images, for example, of such area and identifying locations of lymph nodes therein, conducting biopsies of sentinel lymph nodes responding to said marker compound to determine the malignant tumor status of such sentinel lymph nodes, subsequently operating to remove both the malignant tumor and tumour-cell invaded sentinel lymph nodes of the lymph basin drainage area of the malignant tumor, and subsequently optionally subjecting the area containing any remaining said lymph nodes and ducts to an oscillating electromagnetic field thereby inducing heat in said magnetic particles containing marker compound, to break down any remaining malignant tumor cells in the lymph system in that area.

Preferably the magnetic particles containing marker compound consists of Co⁺², Co⁺³, Fe⁺², Fe⁺³ and/or their oxides or salts;

Preferably the magnetic particles containing marker compound contains such magnetic particles in a size from less than 5 nm up to 500 μm.

Preferably the magnetic particles containing marker compound is either uncoated or coated with a therapeutically acceptable coating substance.

Preferably the magnetic particles containing marker compound is in the form of powder, or in unstable and stabile suspensions (magnetic fluids).

Preferably the magnetic particles containing material will comprises particles which are characterized by high opacifying ability and may be used successfully as contrast in ultra-sonography and X-ray investigations.

Preferably the marker compound may be prepared on a base of an apyrogenic physiological-isotonic salt solution, to be more suitable for clinical application.

The various features of novelty which characterize the invention are pointed out with more particularity in the claims annexed to and forming a part of this disclosure. For a better understanding of the invention, its operating advantages and specific objects attained by its use, reference should be made to the accompanying drawings and descriptive matter in which there are illustrated and described preferred embodiments of the invention.

### IN THE DRAWINGS

FIG. 1 is a schematic illustration of a malignant tumor and surrounding lymph systems;

FIG. 2 is a schematic illustration showing the injection of a marker compound;

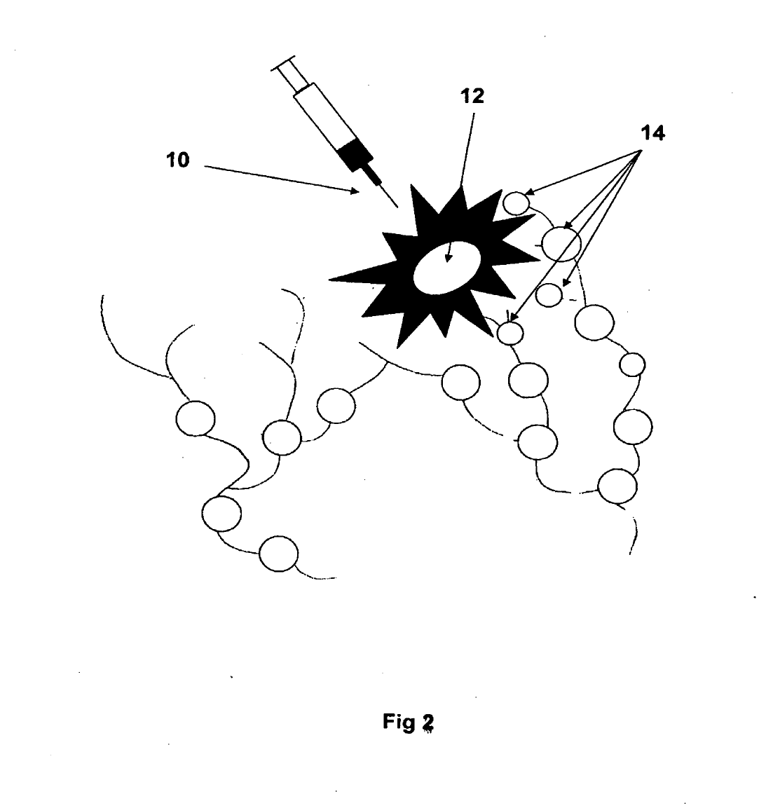

FIG. 3 is a schematic illustration showing the resulting image, and the trepan biopsy step, or biopsy step;

FIG. 4 is a schematic illustration showing the surgical removal of the tumour, and the sentinel lymph nodes;

FIG. 5 is a schematic illustration showing the hyperthermal therapy of the residual lymph system.

### DESCRIPTION OF A SPECIFIC EMBODIMENT

As described above, the invention provides a series of steps for the diagnosis and treatment of malignant tumours, by surgical removal, and treatment of the lymph system.

The invention uses a marker compound containing magnetic particles. Such compounds have two significant functions: First, the compound collects in the lymph system. This enables the lymph system which is draining the region surrounding the tumour to be visually detected and/or imaged. The imaging may be by X-ray, or by MRI mapping, or by ultrasound, for example, although other imaging systems, possibly not yet available, may serve equally well. Secondly the marker compound has the property of remaining in the lymph system for some time even after the surgical operation has been completed. This enables the optional further treatment by hyperthermal methods on lymph tissue in the area of the location surrounding the tumour site. This is achieved by subjecting the area to a fluctuating magnetic field, at appropriate frequencies and strengths, to excite the magnetic particles, and heat those nodes and ducts of the lymph system, containing such magnetic particles, which remain after the surgical operation.

Step 1 Tumour Site Detection:

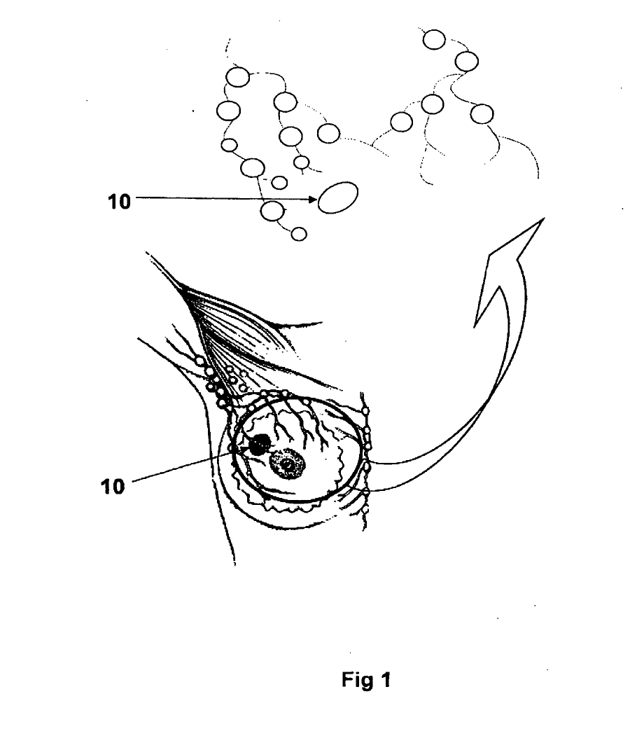

Locate tumour site as shown at 10 in FIG. 1.

Step 2 Sentinel Lymphatic Nodes Detection:

Injection of the marker compound magnetic particles 12, in lymph nodes 14.

The particles will be retained by macrophages in lymph nodes draining the tumour site. This mechanism of particles transport underlies to the new method of sentinel lymph node detection.

Unlike the other vital dyes referred to above, magnetic particles actively fill lymphatic nodes and color them in bold black for a long time (more then 1 week).

It is significant that magnetic particles are not radioactive compounds and they can not be detected by gamma probe detector. However they do provide the possibility of X-ray and/or ultrasound imaging of sentinel lymph nodes making it possible to conduct target trepan-biopsy. High opacifying ability of magnetic particles and retention of bold black color of sentinel lymphatic nodes for a long time significantly facilitates the intraoperating detection and atraumatic performing of lymphadenectomy.

Step 3 Imaging and Biopsy:

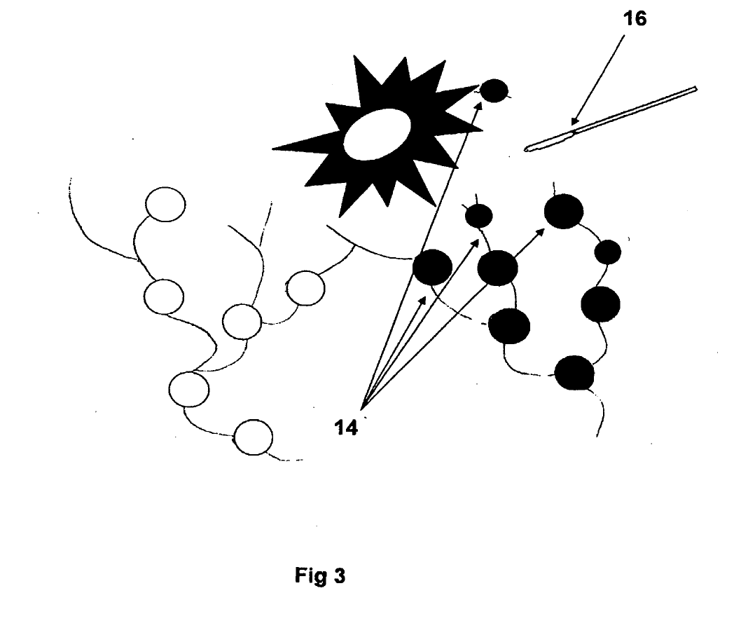

Imaging of lymph nodes 14 by eg. X ray, ultrasound or the like. Then followed by biopsy 16 (FIG. 3), of the sentinel lymph nodes, marked by the marker compound to determine their tumour status.

Step 4 Surgical Removal:

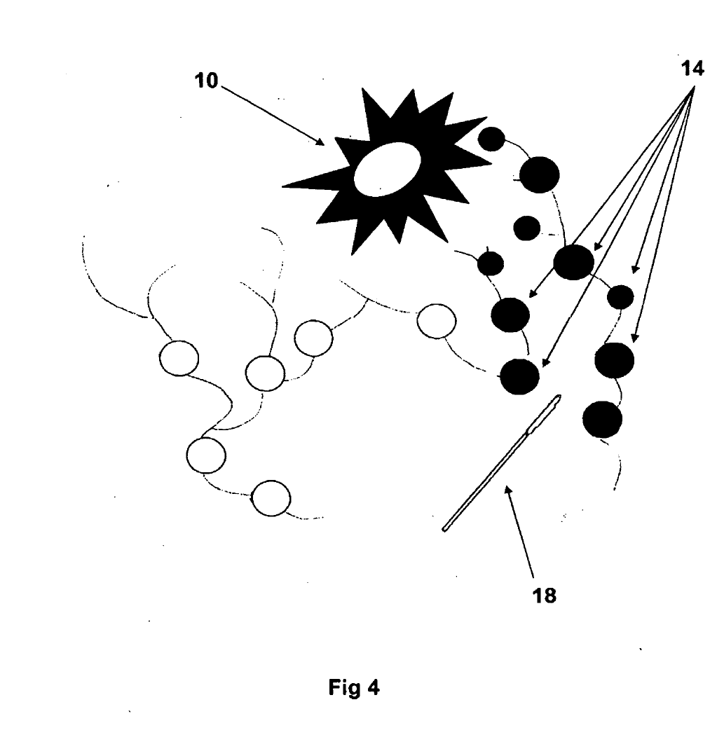

Surgical removal 18 (FIG. 4) of tumour and of lymph nodes of the drainage area having positive tumour status.

Step 5 Hyper-thermal Influence on Metastatic Lesions:

This a further optional step.

It is known that in the case of spreading of metastasis in regional lymphatic nodes, the lymph basin, draining the area of localization of malignant tumor must be removed. Even in the case of ideal surgical intervention is often impossible to remove all the microscopic lymphatic capillaries and nodes which may contain tumor cells. In order to reduce or eliminate the development of secondary metastasis, it may be advisable to provide the chemo and radio therapy.

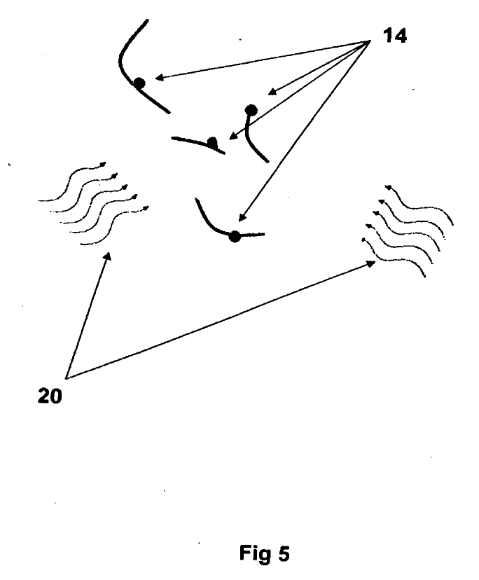

One of the appealing properties of magnetic particles are their self heating ability when placed in a high frequency electromagnetic field 20 (FIG. 5).

In the case of sentinel lymphatic nodes containing tumor cells the use of the magnetic particles described heating effect in postoperative period can act as a therapeutic agent which targets and causes hyper-thermal influence on left after operation local micro lymphatic nodes. It will minimize the potentiality of development of postoperative recurrence and secondary metastatic processes.

Features of the Treatment Method of the Invention:

The method provides a high degree of coloring of lymphatic nodes in bold black for a long time (more then 1 week), rendering it easy to identify them. The method employs X ray and/or ultrasound or other imaging technology, for an indication of lymphatic nodes in preoperative period, thus providing the possibility of conducting target trepan-biopsy, and also biopsy;

The method facilitates the surgical removal of potentially tumor infected sentinel nodes, during the operation.

The method permits treatment by distant hyper-thermal influence on local micro lymphatic nodes in postoperative period, for prevention of development of postoperative recurrence and secondary metastatic processes;

All above mentioned features of the method of the invention contribute to the provision of a safe and accurate method of detection of sentinel lymphatic nodes and performing of target trepan biopsy in preoperative period, discovering and/or removing them during the operation, and treating them in postoperative period in malignant tumors.

Magnetic suspensions, for use in the method can be both unstable suspensions, and also stabilized suspensions. In unstable suspensions the Magnetic particles are not covered by stabilizers. They will stick together and will settle out.

In stabilized suspensions, the Magnetic particles are essentially coated in the stabilizing component. This prevents the particles from sticking together and thus keeps them in suspension.

For usual treatment the solution will be injected in an amount of between about 1 ml and 5 ml.

Suitable components are shown below:

1. Particles: Magnetic particles consists of Co⁺², Co⁺³, Fe⁺², Fe⁺³ and/or their oxides including magnetite [Fe(FeO₂)₂] and/or complexes with Mn, Zn, Cr and etc. in a size from less than 5 nm up to 500 μm.

2. Commonly encountered stabilizers, surfactants: therapeutically acceptable coating substance:

Ionic: complexes

Anionic (based on sulfate, sulfonate or carboxylate anions):
Sodium dodecyl sulfate, ammonium lauryl sulfate, and other alkyl sulfate salts.
Sodium laureth sulfate, also known as sodium lauryl ether sulfate.
Alkyl benzene sulfonate.
Soaps, or fatty acid salts

Cationic (based on quaternary ammonium cations):
Cetyl trimethylammonium bromide, hexadecyl trimethyl ammonium bromide, and other alkyltrimethylammonium salts
Cetylpyridinium chloride
Polyethoxylated tallow amine
Benzalkonium chloride
Benzethonium chloride

Zwitterionic (amphoteric):
Dodecyl betaine
Cocamidopropyl betaine
Coco ampho glycinate

Nonionic:
Alkyl poly(ethylene oxide)
Alkylphenol poly(ethylene oxide)
Copolymers of poly(ethylene oxide) and poly(propylene oxide)
Alkyl polyglucosides, including:
Octyl glucoside
Decyl maltoside

Fatty alcohols:
Cetyl alcohol
Oleyl alcohol

Fatty acids:
Oleic acid

Organic acids:
Citric acid

Proteins:
Albumine

Fatty substances:
Soy Lecithin

Cocamide MEA, cocamide DEA

Polysorbates: Tween 20, Tween 80
Dodecyl dimethylamine oxide

3. Carriers: distilled Water; Apyrogenic (not producing fever) Physiological- isotonic salt solution (9 g NaCl dissolved in 1 liter water. The mass of milliliter of Physiological solution is 1.009 grams. The molecular weight of sodium chloride is approximately 58 g/mole, so 58 g NaCl is 1 mole. Since Physiological solution contains 9 grams NaCl, the concentration is 9 g/L divided by 58 g/mole=0.154 mole/L.).

Suitable Formulations are Shown Below:

Example of magnetic suspensions which is unstable, for use in the method is:

1-50% of the magnetic particles containing marker compound consists of Co⁺², Co⁺³, Fe⁺², Fe⁺³ and/or their oxides including magnetite [Fe(FeO₂)₂] and/or complexes with Mn, Zn, Cr and etc. in a size from less than 5 nm up to 500 μm.

99-50% of the apyrogenic (not producing fever) Physiological- isotonic salt solution (9 g NaCl dissolved in 1 liter water. The mass of milliliter of Physiological solution is 1.009 grams. The molecular weight of sodium chloride is approximately 58 g/mole, so 58 g NaCl is 1 mole. Since Physiological solution contains 9 grams NaCl, the concentration is 9 g/L divided by 58 g/mole=0.154 mole/L.).

Example of magnetic suspensions which is stable, for use in the method is:

0.5-10% of the magnetic particles containing marker compound consists of Co⁺², Co⁺³, Fe⁺², Fe⁺³ and/or their oxides including magnetite [Fe(FeO₂)₂] and/or complexes with Mn, Zn, Cr and etc. in a size from less than 5 nm up to 500 μm.

3-20% of stabilizers, surfactants- therapeutically acceptable coating substance.

96.5-70% of the apyrogenic (not producing fever) Physiological-isotonic salt solution (9 g NaCl dissolved in 1 liter water. The mass of milliliter of Physiological solution is 1.009 grams. The molecular weight of sodium chloride is approximately 58 g/mole, so 58 g NaCl is 1 mole. Since Physiological solution contains 9 grams NaCl, the concentration is 9 g/L divided by 58 g/mole=0.154 mole/L.).

Clinical Case Report N1

It is the clinical case report of 30 year old woman with a nodule in the upper outer quadrant of the left breast. Sade nodule had been discovered six month previously and had gradually increased in size during the intervening period. Clinical examination had showed the presence of 4 cm. in diameter, non-painful, hard, mobile nodule with well shaped margins, smooth surface. The histopathological examination of the piece of tissue received by performing of trepan biopsy of the tumor showed the presence of a secretory carcinoma.

The day before the operation the sentinel lymph nodes were identified with the use of innovative preparation on the base of magnetite nano-particles. 3 ml. of said preparation was injected around of malignant tumor. Sentinel Lymph nodes had been detected in axillary basin by Ultrasound and X-ray examination of the patient, performed after 15-20 min. of injection of said preparation. The sentinel lymph node trepan biopsy was performed under the “Tele-X-ray” control. The histopathological examination of the piece of tissue received by performing of said trepan biopsy showed the metastatic cancer cells similar to those of the primary tumor cells.

An upper outer quadrantectomy of the left breast was performed, together with total axillary lymphadenectomy under general anaesthetic. High rate of coloring of lymphatic nodes in bold black for a long time helped in the intraoperative identification of them. Said nodes showed up as bold black and were therefore removable without any difficulty.

Clinical Case Report N2

It is the clinical case report of 64 year old man with a pink nodular lesion of the right upper arm. The histopathological examination of the piece of tissue received by biopsy of the arm lesion shown existence the nodular melanoma.

The day before the operation the sentinel lymph nodes were identified with the use of innovative preparation on the base of magnetite nano-particles. 3 ml. of said preparation was injected around of malignant tumor. Lymph nodes had been detected in axillary basin by Ultrasound and X-ray examination of the patient, performed after 15-20 min. of injection of said preparation. The sentinel lymph node trepan biopsy was performed under the “Tele-X-ray” control. The histopathological examination of the piece of lymph node tissue received by performing of said trepan biopsy showed the existence of metastatic melanoma.

Wide local excision with 2-cm margins was performed, together with total axillary lymphoadectomy under general anaesthetic. High rate of coloring of lymphatic nodes in bold black for a long time helps in the intraoperative identification of them. Said nodes showed up as bold black and were therefore removable without any difficulty.

The term “biopsy” as used herein, includes both surgical removal of one or more sentinel lymph nodes, for analysis, and also trepan biopsy methods, in which a needle is inserted into a targeted sentinel lymph node and a small portion of that sentinel lymph node is removed and analyzed.

The foregoing is a description of a preferred embodiment of the invention which is given here by way of example only. The invention is not to be taken as limited to any of the specific features as described, but comprehends all such variations thereof as come within the scope of the appended claims.

### LIST OF REFERENCES

1. Cabanas R M. An approach for the treatment of penile carcinoma. Cancer. 1977; 39:456-66.

2. Morton D L, Wen D R, Wong J H, Economou J S, Cagle L A, Storm F K, Foshag L J, Cochran A J. Technical details of intraoperative lymphatic mapping for early stage melanoma. Arch. Surg. 1992; 127:392-9.

3. Alex J C, Krag D N. Gamma-probe guided localization of lymph nodes. Surg. Oncol. 1993; 2:137-43.

4. Giuliano A E, Kirgan D M, Guenther J M, Morton D L. Lymphatic mapping and sentinel lymphadenectomy for breast cancer. Ann Surg 1994; 220:391-8.

5. Bergkvist L, Frisell J, Liljegren G, Celebioglu F, Damm S, Thorn M. Multicenter study of detection and false-negative rates in sentinel node biopsy for breast cancer. Br. J. Surg. 2001; 88:1644-8.

6. Krag D N, Weaver D L, Alex J C, et al. Surgical resection and radiolocalization of the sentinel lymph node in breast cancer using a gamma probe. Surg. Oncol. 1993; 2:335-9.

7. Albertini J J, Lyman G H, Cox C E, et al. Lymphatic mapping and sentinel node biopsy in the patient with breast cancer. JAMA 1996; 276:1818-22.

8. McMasters K M, Tuttle T M, Carlson D J, et al. Sentinel lymph node biopsy for breast cancer: a suitable alternative to routine axillary dissection in multi-institutional practice when optimal technique is used. Clin. Oncol. 2000; 18:2560-6.

9. Cox C E. Lymphatic mapping in breast cancer: combination technique. Ann. Surg. Oncol. 2001; 8 (9 Suppl): 67-70.

10. Derossis A M, Fey J, Yeung H, et al. A trend analysis of the relative value of blue dye and isotope localization in 2000 consecutive cases of sentinel node biopsy for breast cancer. J. Am. Coll. Surg. 2001; 193:473-8.

11. Noguchi M. Sentinel lymph node biopsy and breast cancer. Br J Surg 2002; 89:21-34.

12. McCarter M D, Yeung H, Yeh S, Fey J, Borgen P I, Cody H S III. Localization of the sentinel node in breast cancer: identical results with same-day and day-before isotope injection. Ann. Surg. Onco. 2001; 8:682-6.

13. Solorzano C C, Ross M I, Delpassand E, et al. Utility of breast sentinel lymph node biopsy using day-before-surgery injection of high-dose ^{99m}Tc-labeled sulfur colloid. Ann. Surg. Oncol. 2001; 8:821-7.

14. Nathanson S D, Wachna D L, Oilman D, Karvelis K, Haystad S, Ferrara J. Pathways of lymphatic drainage from the breast. Ann. Surg. Oncol. 2001; 8:837-43.

15. Cox C E, Haddad F, Bass S, et al. Lymphatic mapping in the treatment of breast cancer. Oncology 1998; 12:1283-92.

What is claimed is:

1. A method of diagnosis and treatment of malignant tumours and comprising the steps of:
locating an area containing lymph systems including sentinel lymph nodes and lymph ducts which drain the area around a tumour;
injecting a magnetic particle-containing marker compound in the region around said tumour, said compound migrating into said sentinel lymph nodes and lymph system which drain the area around a tumour;
making images of such area, by methods selected from the group comprising, X-ray, Ultrasound and the like, and identifying locations of sentinel lymph nodes therein;
conducting biopsies of sentinel lymph nodes responding to said marker compound to determine the tumour status of such sentinel lymph nodes;
operating to remove both the malignant tumour and tumour-invaded lymph nodes.

2. The method as claimed in claim 1 and including the steps of detecting tumour cells in one or more sentinel lymph nodes, and thereafter providing the biopsy of said sentinel lymph nodes, surgically removing colored lymph nodes and lymph system which drain the area around a tumour during removal of the tumour and subsequently subjecting the area around said tumour and containing any remaining said lymph nodes and ducts to an hyperthermal treatment by an oscillating electromagnetic field thereby inducing heat in said magnetic particles containing marker compound, to break down any potentially remaining tumour cells in the said remained micro lymph nodes in that area.

3. The method as claimed in claim 1 wherein the magnetic particles containing marker compound consists of Co⁺², Co⁺³, Fe⁺², Fe⁺³ and/or their oxides including magnetite [Fe(FeO₂)₂] and/or complexes with Mn, Zn, Cr .

4. The method as claimed in claim 1 wherein the magnetic particles containing marker compound contains such magnetic materials selected from particles in the size range from less than 5 nm up to 500 μm.

5. The method as claimed in claim 1 wherein the magnetic particles containing marker compound is selected from the group comprising an uncoated marker compound, and a marker compound coated with a therapeutically acceptable coating substance.

6. The method as claimed in claim 1 wherein the magnetic particles containing marker compound is selected from the group comprising powder, and unstable suspension and stable suspensions (magnetic fluids).

7. The method as claimed in claim 1 wherein the magnetic particles containing material will comprises particles which are characterized by a black colour having high opacifying ability

8. The method as claimed in claim 6 wherein said therapeutically acceptable compound is selected from the group comprising therapeutically acceptable, stabilizers, surfactants.

9. The method as claimed in claim 8 wherein the surfactants are selected from the group comprising ionic and nonionic surfactants: Oleic acid, Citric acid, Soy lecithin, Polysorbate-80 (trade name Tvin 80.

10. The method as claimed in claim 1 wherein said marker compound includes a conductive enhancer.

11. The method as claimed in claim 10, wherein said conductive enhancer is Apyrogenic (not producing fever) Physiological-isotonic salt solution (9 g NaCl dissolved in 1 liter water. The mass of milliliter of Physiological solution is 1.009 grams. The molecular weight of sodium chloride is approximately 58 g/mole, so 58 g NaCl is 1 mole. Since Physiological solution contains 9 grams NaCl, the concentration is 9 g/L divided by 58 g/mole=0.154 mole/L.).

12. A compound for marking sentinel lymph nodes in a drainage area around a malignant tumour and comprising; magnetic particles containing marker compound consists of Co⁺², Co⁺³, Fe⁺², Fe⁺³ and/or their oxides including magnetite [Fe(FeO₂)₂] and/or complexes with Mn, Zn, Cr , and a carrier.

13. The compound as claimed in claim 12 and wherein the magnetic particles containing marker compound contains such magnetic materials selected from particles in the size range from less than 5 nm up to 500 μm.

14. The compound as claimed in claim 13 wherein the magnetic particles containing material will comprises particles which are characterized by a black colour having high opacifying ability

15. The compound as claimed in claim 14 wherein the magnetic particles containing marker compound is selected from the group comprising powder, and unstable suspension and stable suspensions (magnetic fluids).

16. The compound as claimed in claim 15 wherein said therapeutically acceptable compound is selected from the group comprising therapeutically acceptable, stabilizers, surfactants and wherein the surfactants are selected from the group comprising ionic and nonionic surfactants: Oleic acid, Citric acid, Soy lecithin, Polysorbate-80 (trade name Tvin 80).

17. The compound as claimed in claim 16 including a conductive enhancer comprising Apyrogenic (not producing fever) Physiological- isotonic salt solution (9 g NaCl dissolved in 1 liter water. The mass of milliliter of Physiological solution is 1.009 grams. The molecular weight of sodium chloride is approximately 58 g/mole, so 58 g NaCl is 1 mole. Since Physiological solution contains 9 grams NaCl, the concentration is 9 g/L divided by 58 g/mole=0.154 mole/L.)
